(19) United States
(12) Patent Application Publication
(10) Pub. No.: US 2005/0164181 A1
(43) Pub. Date: Jul. 28, 2005

(54) Nanostructure, in particular for analysing individual molecules

(76) Inventor: Thomas Bricson, Kista (SE)

Correspondence Address: SUTHERLAND ASBILL & BRENNAN LLP, 999 PEACHTREE STREET, N.E., ATLANTA, GA, 30309, US

(21) Appl. No.: 10/496,726

(22) PCT Filed: Nov. 27, 2002
(86) PCT No.: PCT/EP02/13390

Related U.S. Application Data
(60) Provisional application No. 60/333,450, filed on Nov. 27, 2001.

(51) Int. Cl. C12Q 1/68; C12M 1/34

(52) U.S. Cl. 435/6; 435/287.2; 435/288.5; 435/288.7

(57) ABSTRACT

The invention concerns a nanostructure and a method for analysing or synthesising a small number of molecules or single molecules and in particular for sequencing single molecules of nucleic acids.

The invention concerns a nanostructure and its use for synthesizing and analysing molecules, especially single molecules e.g. for sequencing single molecules of nucleic acids.

The sequencing of the human genome composed of about 3×10⁹ bases or of the genome of other organisms and the determination and comparison of individual sequence variants requires the provision of sequencing methods which are, on the one hand, rapid and which on the other hand, can be used routinely and at low costs. Major attempts have been made in recent years to accelerate the current sequencing methods e.g. the enzymatic chain termination method according to Sanger et al. (Proc. Natl. Acad. Sci. USA 74 (1977) 5463), in particular by automation (Adams et al., Automated DNA Sequencing and Analysis (1994), New York, Academic Press). Thus at present a maximum of 500 000 bases can be determined per day with a sequencer (Mega Bace from Applied Biosystems). Nevertheless, conventional sequencing methods are less suitable for some applications.

New approaches for overcoming the limitations of conventional sequencing methods have been developed in recent years which include sequencing by scanning-tunnel microscopy (Lindsay and Phillip, Gen. Anal. Tech. Appl. 8 (1991), 8-13), by highly parallelized capillary electrophoresis (Huang et al., Anal. Chem. 64 (1992), 2149-2154; Kambara and Takahashi, Nature 361 (1993), 565-566), by oligonucleotide hybridization (Drmanac et al., Genomics 4 (1989), 114-128; Khrapko et al., FEBS Let. 256 (1989), 118-122; Maskos and Southern, Nucleic Acids Res. 20 (1992), 1675-1678 and 1679-1684) and by matrix-assisted laser desorption/ionization mass spectroscopy (Hillenkamp et al., Anal. Chem. 63 (1991), 1193A-1203A).

Another approach is single molecule sequencing (Dörre et al., Bioimaging 5 (1997), 139-152) in which the sequence of nucleic acids is determined by successive enzymatic degradation of fluorescently-labelled single-stranded DNA molecules and detection of the sequentially released monomer molecules in a microstructured channel in which the monomer molecules are electroosmotically transported by pumps. The advantage of this method is that in each case only a single molecule of the target nucleic acid is sufficient to carry out a sequence determination.

However, a disadvantage of the method described by Dörre et al. is that the sequentially released monomer molecules can interact with the walls of the microstructures which can lead to considerable problems in the evaluation.

Methods and devices for controlling laminar fluid currents in a flow cell are described in WO 99/36766. Discrete sensor areas are generated that can be used to detect analytes in a sample liquid.

Methods and devices for single molecule sequencing are proposed in PCT/EP01/07460. For this a system of microchannels that are in fluidic communication is used where liquids are transported through the microchannels by means of a hydrodynamic flow.

The object of the present invention was to provide a device and a method for synthesizing and analysing molecules, in particular for the single molecule sequencing of nucleic acids which represents a further improvement over the prior art and in particular enables a simplified analysis.

This object is achieved by providing a device comprising:
(a) a system of at least partially optically transparent microchannels that are in fluidic communication comprising
   (i) a first microchannel for introducing a first fluid flow comprising a carrier particle with at least one molecule immobilized thereon,
   (ii) at least one second microchannel for introducing at least one second fluid flow comprising a reactant for the immobilized molecule,
   (iii) optionally at least one third microchannel for introducing at least one third fluid flow,
   (iv) at least one fourth microchannel wherein the microchannels (i), (ii) and (iii) lead into the fourth microchannel and at least the first and second fluid flows flow essentially without mixing at least in one section of the fourth micro channel,
(b) means for holding the carrier particle at a predetermined position in the area of the section of the fourth microchannel in which the fluid flows flow essentially without mixing,
(c) means for contacting the retained carrier particle with the second fluid flow containing the reactant and
(d) optionally means for detecting a reaction of the immobilized molecule with the reactant.

The device according to the invention is based on the principle that a carrier having several microchannels that are in fluidic communication is provided where at least two and preferably three or four or more microchannels are used to introduce different fluid flows into the carrier. These microchannels join a further microchannel in which the immobilized molecule is reacted with one or more reactants. A carrier particle loaded with one or more immobilized molecules is introduced into the device in a first fluid flow through a first microchannel. Second fluid flows which each contain reactants for the immobilized molecule are introduced into the device in one or more second microchannels. Preferably one or more third microchannels are provided that are used to introduce one or more third fluid flows and these third fluid flows can for example contain buffer solutions and be used for example to separate the first fluid flow from one or more second fluid flows. The geometry of the device is designed such that the fluid flows that flow into the fourth microchannel are essentially not intermixed at least in one section of the fourth microchannel and in particular in the area of the discharge zone and the individual fluid flows preferably flow in a laminar flow.

After being introduced into the device, the loaded particle is held by suitable means at a first predetermined position in the fourth microchannel e.g. in the area of the discharge zone. At this position the retained particle can be contacted by suitable means with one or more second fluid flows which contain free reactants for the immobilized molecule such that a reaction can occur between the immobilized molecule and one or more free reactants. The presence of separate and preferably laminar fluid flow zones in the fourth microchannel enables a specific control of the contact between the molecule and reactant. The reaction can preferably be detected downstream in the fourth microchannel.

In a first embodiment the retained carrier particle is transported within the fourth microchannel from the first predetermined position to a second predetermined position. This transport occurs from the zone of the first fluid flow into the zone of a second fluid flow and preferably over the zone of a third fluid flow. Furthermore it is preferred that the transport is essentially transverse to the direction of flow.

In another embodiment the retained carrier particle does not have to be transported but rather the carrier particle is contacted with the second fluid flow by changing the flow conditions in the fourth microchannel e.g. by changing the flow velocities in one or more microchannels such that the trapped carrier particle, which is initially in a zone corresponding to the first fluid flow, comes into a zone in which it comes into contact with the second fluid flow by changing the flow conditions. Of course both of the aforementioned embodiments can also be combined with one another.

In addition to the first and the one or more second microchannels which transport the carrier particle with the immobilized molecule and the reactant or reactants into the device, preferably one or more third microchannels are additionally provided which for example transport inert fluid flows such as buffer solutions into the device. In this case the third microchannel or third microchannels can be arranged such that one or more third fluid flows are generated in the fourth microchannel which are located between the first and second fluid flows.

The device according to the invention can be used to analyse molecules. When carrier particles are used on which only a single molecule is immobilized, the device is suitable for carrying out single molecule analyses for example to characterize biomolecules. A particularly preferred field is the sequencing of nucleic acids. In addition the device can also be used to synthesize molecules in which case an initial product is immobilized on the carrier particle which is simultaneously or sequentially contacted with one or more reaction partners in the device according to the invention. In this manner it is for example possible to synthesize organic compounds e.g. complex organic compounds or biopolymers such as peptides, polypeptides, nucleic acids and nucleic acid analogues. In this connection the device is also especially suitable for synthesizing single molecules.

When the device is used for analytical applications e.g. for nucleic acid sequencing, it advantageously contains a means for detecting a reaction of the immobilized molecule with the reactant such as a suitable detector. If the device is used for syntheses, it advantageously contains means for isolating the molecule synthesized on the carrier particle optionally after cleavage from the carrier particle.

In addition the device can contain means for passing fluid flows into the microchannels, means for discharging fluid flows from the microchannels and reservoirs for first, second and optionally third fluid flows. Furthermore it preferably contains automatic manipulation devices, heating or cooling devices such as Peltier elements, means for sorting carrier particles or/and products of synthesis, reagents and electronic control and evaluation devices.

Another subject matter of the invention is a method for carrying out a reaction between an immobilized molecule and a free reactant e.g. for single molecule analysis and in particular for single molecule sequencing comprising:
(a) providing a carrier particle with a molecule immobilized thereon,
(b) introducing the carrier particle into a system of at least partially optically transparent microchannels that are in fluidic communication where the device comprises:
   (i) a first microchannel for introducing a first fluid flow comprising the carrier particle,
   (ii) at least one second microchannel for introducing at least one second fluid flow comprising a reactant for the immobilized molecule,
   (iii) optionally at least one third microchannel for introducing a third fluid flow,
   (iv) at least one fourth microchannel wherein the microchannels (i), (ii) and (iii) lead into the fourth microchannel and at least the first and second fluid flows flow essentially without mixing at least in one section of the fourth microchannel,
(c) holding the carrier particle at a predetermined position in the area of the section of the fourth microchannel in which the fluid flows flow essentially without mixing,
(d) contacting the retained carrier particle with at least a second fluid flow containing the reactant and
(e) optionally detecting the reaction between the immobilized molecule and the reactant.

The method according to the invention is preferably a sequencing method in which a single nucleic acid molecule immobilized on a carrier is examined. However, the method is suitable for other analytical and synthetic reactions which can be carried out with a small number of for example up to 1000 molecules or even only with single molecules. It is particularly preferred to carry out analytical reactions that can be detected within the device by optical methods.

The carrier particle used for the method has a size which enables it to be moved in microchannels and held at a desired position within a sequencing device. The particle size is preferably in the range of 0.5 to 10 μm and particularly preferably 1 to 3 μm. Examples of suitable materials for carrier particles are plastics such as polystyrene, polymethylmethacrylate, polypropylene, polycarbonate or copolymers thereof, glass, quartz, metals or semi-metals such as silicon, metal oxides such as silicon dioxide or composite materials that contain several of the aforementioned components. Optically transparent carrier particles that are for example made of plastics or glass, particles made of silicon or particles with a plastic core and a silicon or silicon dioxide shell are particularly preferably used.

Nucleic acid molecules are preferably immobilized on the carrier particles via their 5′ ends. The nucleic acid molecules can be bound to the carrier by covalent or non-covalent interactions. For example the binding of polynucleotides to the carrier can be mediated by high affinity interactions between the partners of a specific binding pair e.g. biotin/streptavidin or avidin, hapten/anti-hapten antibody, sugar/lectin etc. Thus biotinylated nucleic acid molecules can be coupled to streptavidin-coated carriers. Alternatively the nucleic acid molecules can also be bound adsorptively to the carrier. Thus nucleic acid molecules modified by the incorporation alkanethiol groups can be bound to metallic carriers e.g. gold carriers. Yet another alternative is covalent immobilization in which the binding of the polynucleotides can be mediated by reactive silane groups on a silica surface.

Carrier particles to which only a single molecule e.g. a nucleic acid molecule is bound are used for the method according to the invention. Such carrier particles can be produced by contacting the molecules to be analysed, e.g. the nucleic acid molecules that are to be sequenced, at a molar ratio of preferably 1:5 to 1:20, e.g. 1:10 with the carrier particles under conditions where an immobilization of the molecules on the carrier takes place. The resulting carrier particles are then sorted e.g. on the basis of fluorescent marker groups on the molecules and separated from particles to which no molecule is bound. This sorting and separation can for example be carried out by the methods described in Holm et al. (Analytical Methods and Instrumentation, Special Issue μTAS 96, 85-87), Eigen and Rigler (Proc. Natl. Acad. Sci. USA 91 (1994), 5740-5747) or Rigler (J. Biotech. 41 (1995), 177-186) which include a detection with a confocal microscope.

The nucleic acid molecules bound to a carrier e.g. DNA molecules or RNA molecules, can be present in a single-stranded form or double-stranded form. In the case of double-stranded molecules it must be ensured that labelled nucleotide building blocks can only be cleaved from a single strand. In the case of nucleic acid strands to be sequenced essentially all e.g. at least 90%, preferably at least 95% of all nucleotide building blocks of at least one base type carry a fluorescent marker group. Preferably essentially all nucleotide building blocks of at least two base types, for example two, three or four base types, carry a fluorescent label and each base type advantageously carries a different fluorescent marker group. Such labelled nucleic acids can be produced by enzymatic primer extension on a nucleic acid template using a suitable polymerase e.g. a DNA polymerase such as a DNA polymerase from Thermococcus gorgonarious or other thermostable organisms (Hopfner et al., PNAS USA 96 (1999), 3600-3605) or a mutated Taq polymerase (Patel and Loeb, PNAS USA 97 (2000), 5095-5100) using fluorescently-labelled nucleotide building blocks. The labelled nucleic acid strands can also be produced by amplification reactions e.g. PCR. Thus in an asymmetric PCR, amplification products are formed where only a single strand contains fluorescent labels. Such asymmetric amplification products can be sequenced in a double-stranded form. Nucleic acid fragments are produced by symmetric PCR where both strands are fluorescently labelled. These two fluorescently labelled strands can be separated and immobilized separately in a single-stranded form on the carrier particles such that the sequence of one or both complementary strands can be determined separately. Alternatively one of the two strands can be modified at the 3′ end e.g. by incorporation of a PNA clamp such that monomer building blocks can no longer be cleaved. In this case double-stranded sequencing is possible.

A sequence identifier i.e. a labelled nucleic acid of known sequence can optionally be attached to the nucleic acid strand to be examined e.g. by enzymatic reaction with ligase or/and terminal transferase such that at the start of sequencing a known fluorescence pattern is firstly obtained and only subsequently the fluorescence pattern corresponding to the unknown sequence to be examined.

The nucleic acid template whose sequence is to be determined can for example be selected from DNA templates such as genomic DNA fragments, cDNA molecules, plasmids etc. and also from RNA templates such as mRNA molecules.

The fluorescent marker groups can be selected from known fluorescent marker groups for labelling biopolymers, e.g. nucleic acids, such as fluorescein, rhodamine, phycoerythrin, Cy3, Cy5 or derivatives thereof etc.

Step (b) of the method comprises introducing a loaded carrier particle into a device according to the invention e.g. a sequencing device.

The introduction of the carrier particle through the first microchannel and the introduction of further fluid flows through the second and third microchannels can be carried out by hydrodynamic or/and electroosmotic flow. A hydrodynamic flow is preferably used. The flow in the inlet channels can be controlled together or separately by using suitable means e.g. pumps, valves or/and gravitation-controlled delivery. The diameter of the first, second and third microchannels can be the same or different and is for example in the range of 5 to 500 μm, particularly preferably in the range of 10 to 100 μm and most preferably 25-75 μm.

The carrier particle can be held in the discharge area of the fourth microchannel with the aid of a trapping laser, e.g. an IR laser, according to process step (c). Such methods are for example described by Ashkin et al., (Nature 330 (1987), 24-31) and Chu (Science 253 (1991), 861-866).

The carrier particle is preferably held by an automated process. For this purpose the carrier particles are passed through the first microchannel during which they pass a detection element which activates the trapping laser. After trapping and relocation into the fluid flow containing a reactant, the reaction on the immobilized carrier particle is then carried out. In the method according to the invention it is not necessary to wash away remaining carrier particles since the trapped carrier particle is relocated from the first fluid flow into a second fluid flow by active transport or/and by changing the flow conditions. Hence it is also possible to trap a further carrier particle as soon as the first retained carrier particle has been removed from the first fluid flow. This considerably increases the speed of the method.

The sequencing reaction of the method according to the invention comprises successive cleavage of single nucleotide building blocks from the immobilized nucleic acid molecules. They are preferably cleaved enzymatically using an exonuclease whereby single strand or double strand exonucleases that degrade in the 5′→3′ direction or 3′→5′ direction can be used depending on the type of immobilization of the nucleic acid strands on the carrier. T7 DNA polymerase, E. coli exonuclease I or E. coli exonuclease II are particularly preferably used as exonucleases.

The reaction products e.g. the nucleotide building blocks released by the cleavage reaction are then passed through the fourth microchannel e.g. by means of a hydrodynamic or/and electroosmotic flow and are preferably determined during the flow through the fourth microchannel. A hydrodynamic flow is preferably used which enables an increase of the flow velocity which in turn increases the probability of detecting a reaction product. Furthermore, the hydrodynamic flow which is for example generated by a sucking action or applying pressure, reduces the occurrence of wall effects compared to electroosmotic pumps. The hydrodynamic flow preferably has a parabolic flow profile i.e. the flow velocity is at a maximum in the centre of the fluid flow and decreases to a minimal velocity towards the edges as a parabolic function. The maximum flow velocity is preferably in the range of 1 to 50 mm/s and particularly preferably in the range of 5 to 10 mm/s. The diameter of the fourth microchannel in the area of the section in which the fluid flows flow essentially without mixing is preferably 5 to 1000 μm, particularly preferably 20 to 500 μm.

In some cases it may be preferable for the fourth microchannel to have a larger diameter than the first, second and third microchannels. However, this is not absolutely necessary. Thus for example the flow velocity can be specifically controlled by local constrictions or/and widening. For example the flow velocity can be increased by constricting the channel and the flow velocity can be specifically decreased by widening.

The identification of the reaction products e.g. of fluorescently-labelled nucleotide building blocks according to step (e) of the method according to the invention can be carried out using any measuring method e.g. with spatially-resolved or/and time-resolved fluorescence spectroscopy which is able to detect fluorescent signals down to a single photon count in a very small volume element such as the one present in a microchannel.

For example the detection can be carried out by means of confocal single molecule detection such as by fluorescence correlation spectroscopy in which case a very small, preferably confocal volume element such as 0.1×10⁻¹⁵ to 20×10⁻¹² l of the sample liquid flowing through the microchannel is exposed to excitation light of a laser which excites the receptors present in this measuring volume to emit fluorescence light, the fluorescence light emitted from the measuring volume being detected by means of a photodetector, and a correlation is established between the change over time of the measured emission and the relative flow velocity of the molecules involved such that single molecules can be identified in the measuring volume at an appropriate dilution. Reference is made to the disclosure of the European Patent 0 679 251 with regard to details of the procedure and details of the devices used for the detection. Confocal single molecule determination is also described by Rigler and Mets (Soc. Photo-Opt. Instrum. Eng. 1921 (1993), 239 ff.) and Mets and Rigler (J. Fluoresc. 4 (1994) 259-264).

Alternatively or in addition the detection can also be by means of a time-resolved decay measurement, a so-called time gating, as described for example by Rigler et al., “Picosecond Single Photon Fluorescence Spectroscopy of Nucleic Acids”, in: “Ultrafast Phenomenes”, D. H. Auston, Springer Publ. 1984. In this case the fluorescence molecules are excited within a measuring volume and subsequently—preferably at a time interval of ≧100 ps—a detection interval is opened on the photo-detector. This enables background signals generated by Raman effects to be kept sufficiently small to allow an essentially interference-free detection.

In a particularly preferred embodiment the detection uses a laser device which has a diffraction element or a phase-modulating element in the optical path of the laser which, optionally in combination with one or more optical imaging elements, is configured to generate a diffraction pattern from the laser beam in the form of a linear or two-dimensional array of focal areas in the microchannel, the optical arrangement being set up to form an image for each focal area that is confocal for the fluorescence detection by the photodetector arrangement. In a further preferred embodiment the detection device is integrated in two opposing sides of bounding walls of the microchannel where one wall has an array of laser elements emitting into the microchannel as a fluorescence excitation light source and the other has an array of photo detector elements which are each arranged opposite to the laser elements as fluorescent light detectors. These two embodiments are disclosed in detail in the Patent Application DE 100 23 423.2.

The probability of detecting nucleotide building blocks can be increased and thus the sensitivity can be improved by a hydrodynamic flow profile in the fluid flows of the first, second and third microchannels as well as in the fluid partial flows of the fourth microchannel of the sequencing device. The hydrodynamic flow can be adjusted and controlled by suitable control devices e.g. by controllable pumps or/and by special geometric design. Electrophoretic and electroosmotic methods can also be used to transport reagents in the sequencing device in addition to hydrodynamic flow. The method according to the invention also allows the parallel sequencing of several carrier particle-bound nucleic acid molecules each in different microchannel systems that are preferably arranged in parallel.

In a particularly preferred embodiment the nucleic acid coupled to a carrier particle is essentially held in the middle of a fluid partial flow, the cleaved fluorescently-labelled nucleotides are passed downstream in the laminar flow to a detection volume element which is positioned essentially above the fluid partial flow and in particular over the middle thereof where there is the highest flow velocity. In this connection the flow velocity is preferably so high that the nucleotide arrives and is registered in the detector field irrespective of thermal spreading of flow trajectories by Brownian diffusion. The detector field is kept as small as possible so that the reaction products e.g. the nucleotide bases are completely detected whereas only the smallest possible fraction of the background contamination (ratio of the detector cross-section to the channel or fluid partial flow cross-section) impinges on the detector.

The invention is additionally elucidated by the following figures.

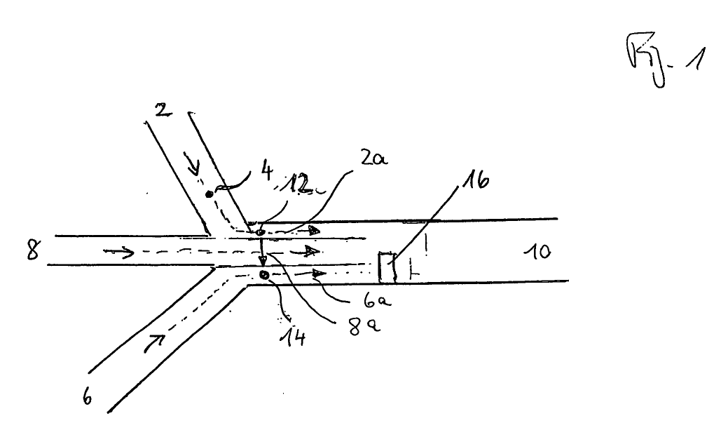

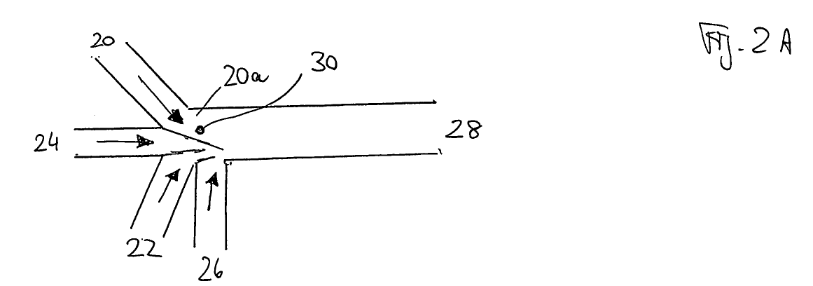

FIG. 1 shows a schematic representation of a preferred embodiment of the device according to the invention. A carrier particle (4) with a molecule immobilized thereon e.g. a nucleic acid molecule, is introduced into the device in a first fluid flow in a first microchannel (2). A second microchannel (6) is used to introduce a second fluid flow which contains a reactant e.g. a reactant provided to degrade the nucleic acid molecule. In addition a third microchannel (8) is provided which is used to introduce a third fluid flow e.g. a buffer solution. The third fluid flow advantageously acts to separate the first and second fluid flow. The first, second and third microchannels (2, 6, 8) load into a fourth microchannel (10). At least in the area where they lead into the fourth microchannel (10), there are separate fluid zones (2a, 6a, 8a) for the fluid flows originating from the microchannels (2, 6, 8) respectively.

The carrier particle (4) introduced through the first microchannel (2) is held in the fourth microchannel (10) at a first predetermined position (12) in the zone of the first fluid flow (2a). The magnetic particle held at the first predetermined position (12) can then, e.g. by using a trapping laser, be transported to a second predetermined position (14) which is in the area of the second fluid flow (6a) which originates from the second microchannel (6). The reaction e.g. the digestion of the nucleic acid molecule immobilized on the carrier particle by the degradation enzyme present in the second fluid flow, preferably an exonuclease, can take place at this second predetermined position (14). The reaction products e.g. the nucleotide building blocks that are sequentially cleaved by the enzymatic digestion, are transported by the fluid flow in the fourth microchannel (10) to a detection element (16), preferably a confocal detection element, and are detected there.

The flow velocity in the system and in particular in the fourth microchannel is adjusted such that the spreading of the migration path of the cleaved nucleotide building blocks caused by Brownian molecular motion is so small that they can be detected with sufficient statistical probability in the detection volume (16).

The diameter of the first, second and third microchannels (2, 6, 8) is preferably in the range of ca. 50 μm. The fourth microchannel (10) preferably has a width of ca. 150 μm and a depth of about 50 μm. The shapes and dimensions of the microchannels can, however, vary considerably provided a flow of several separate fluid flows that is free of mixing is ensured at least in one section of the fourth micro channel.

The sequencing device according to the invention can contain a plurality of the arrangement shown in FIG. 1, e.g. in parallel or/and sequentially, such that it is possible to determine several molecules, e.g. nucleotide sequences, per device in parallel or/and sequentially.

Advantages of the sequencing device according to the invention are in particular that a simple control and separation of several different fluid flows is possible. In addition to the stated use for nucleic acid sequencing, the device can also be used for other analyses e.g. for single molecule detection and also for synthetic processes.

In a variation from the embodiment shown in FIG. 1, devices can also be used in which the third microchannel (8) is absent or in which several second or/and third microchannels are present.

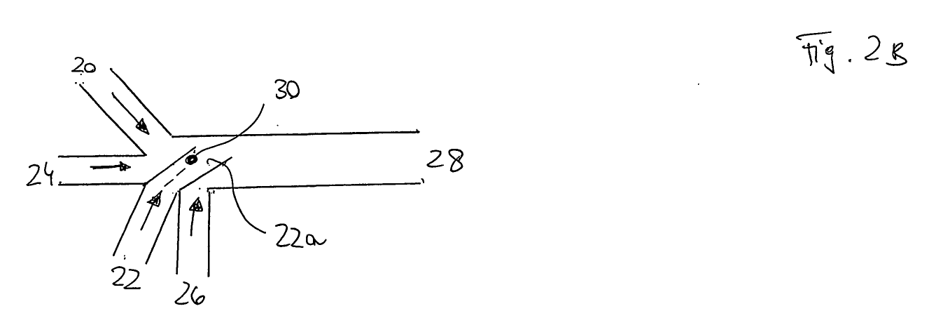

An example of such an embodiment with two third microchannels is shown in FIG. 2. The embodiment of the device according to the invention shown in FIG. 2 allows a detection of the molecule immobilized on the carrier particle without having to transport the carrier particle within the sequencing device after it has been trapped.

For this the carrier particle is firstly introduced into the device through the first microchannel (20) in the setting shown in FIG. 2A and is held at a predetermined position (30) in the fourth microchannel (28) by a trapping laser. The predetermined position (30) is firstly in the area of the fluid flow (20a) coming from the first microchannel (20). The device additionally contains a second microchannel (22) for introducing the reactant as well as two third microchannels (24, 26) for introducing buffer solution.

After the microparticle has been trapped at position (30), the flow conditions are changed in the device e.g. by reducing or switching off the first fluid flow from the first microchannel and by switching on or increasing the fluid flows from a second microchannel (22) and a third microchannel (26). This changes the fluid flow zones belonging to the respective microchannels in the fourth microchannel (28). In the setting shown in FIG. 2B the microparticle held at position (30) thus comes into the area of the second fluid flow (22a) originating from the second microchannel (22) which contains the degradation enzyme.

Of course the embodiments shown in FIG. 1 and FIG. 2 can also be combined with one another. This means that the trapped microparticle can be transported from a first predetermined position to a second predetermined position and also that the fluid flow conditions in the area of the discharge zone in the fourth microchannel can be changed from a trapping setting to an analysis setting.

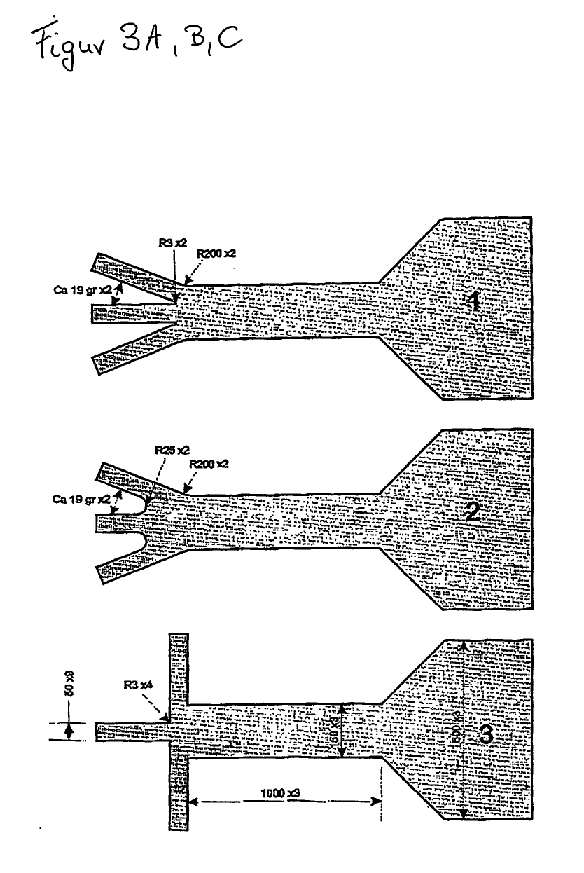

Finally FIGS. 3a, 3b and 3c show concrete embodiments of the device according to the invention that have already been used in practice.

1. Device comprising:
(a) a system of at least partially optically transparent microchannels that are in fluidic communication comprising
(i) a first microchannel for introducing a first fluid flow comprising a carrier particle with at least one molecule immobilized thereon,
(ii) at least one second microchannel for introducing at least one second fluid flow comprising a reactant for the immobilized molecule,
(iii) optionally at least one third microchannel for introducing at least one third fluid flow,
(iv) at least one fourth microchannel wherein the microchannels (i), (ii) and (iii) lead into the fourth microchannel and at least the first and second fluid flows flow essentially without mixing at least in one section of the fourth microchannel,
(b) means for holding the carrier particle at a predetermined position in the area of the section of the fourth microchannel in which the fluid flows flow essentially without mixing,
(c) means for contacting the retained carrier particle with the second fluid flow containing the reactant and
(d) optionally means for detecting a reaction of the immobilized molecule with the reactant.

2. Device as claimed in claim 1,
characterized in that
the first, second and third fluid flows have an essentially laminar flow at least in one section of the fourth microchannel.

3. Device as claimed in claim 1 or 2, additionally comprising means for transporting the retained carrier particle from a first predetermined position in the fourth microchannel to a second predetermined position in the area of a second fluid flow.

4. Device as claimed in one of the claims 1 to 3, additionally comprising means for changing the flow conditions in the fourth microchannel such that the retained carrier particle comes into the area of a second fluid flow.

5. Device as claimed in one of the claims 1 to 4 additionally comprising:
means for passing fluid flows into the microchannels,
means for discharging fluid flows from the microchannels,
reservoirs for the first, second and optionally third fluid flows.

6. Use of a device as claimed in one of the claims 1 to 5 for analysing molecules.

7. Use as claimed in claim 6 for single molecule analysis.

8. Use as claimed in claim 6 or claim 7 for sequencing nucleic acids.

9. Use of a device as claimed in one of the claims 1 to 5 for synthesizing molecules.

10. Use as claimed in claim 9 for synthesizing single molecules.

11. Use as claimed in claim 10 for synthesizing organic compounds or biopolymers.

12. Method for carrying out a reaction between an immobilized molecule and a free reactant comprising:
(a) providing a carrier particle with at least one molecule immobilized thereon,
(b) introducing the carrier particle into a system of at least partially optically transparent microchannels that are in fluidic communication where the device comprises:
(i) a first microchannel for introducing a first fluid flow comprising the carrier particle,
(ii) at least one second microchannel for introducing at least one second fluid flow comprising a reactant for the immobilized molecule,
(iii) optionally at least one third microchannel for introducing at least one third fluid flow,
(iv) at least one fourth microchannel wherein the microchannels (i), (ii) and (iii) lead into the fourth microchannel and at least the first and second fluid flows flow essentially without mixing at least in one section of the fourth microchannel,
(c) holding the carrier particle at a predetermined position in the area of the section of the fourth microchannel in which the fluid flows flow essentially without mixing,
(d) contacting the retained carrier particle with at least one second fluid flow containing the reactant and
(e) optionally detecting the reaction between the immobilized molecule and the reactant.

13. Method as claimed in claim 12,
characterized in that
a carrier particle made of plastic, glass, quartz, metals, semi-metals, metal oxides or a composite material is used.

14. Method as claimed in claim 12 or 13,
characterized in that
a carrier particle made of plastic, glass or silicon or a composite material thereof is used.

15. Method as claimed in one of the claims 12 to 14,
characterized in that
the carrier particle has a diameter of 0.5 to 10 μm.

16. Method as claimed in one of the claims 12 to 15,
characterized in that
a carrier particle is used with a single molecule immobilized thereon.

17. Method as claimed in one of the claims 12 to 16,
characterized in that
a fluid flow for separating the first and second fluid flow is passed through the at least one third microchannel.

18. Method as claimed in one of the claims 12 to 17 for analysing molecules.

19. Method as claimed in one of the claims 12 to 18 for sequencing nucleic acids, comprising:
providing a carrier particle with a nucleic acid molecule immobilized thereon where essentially all nucleotide building blocks of at least one base type in at least one strand of the nucleic acid molecule carry a fluorescent label, successive cleavage of individual nucleotide building blocks from the immobilized nucleic acid molecule, transporting the cleaved nucleotide building blocks through the fourth microchannel and determining the base sequence of the nucleic acid molecule on the basis of the sequence of cleaved nucleotide building blocks.

20. Method as claimed in claim 19,
characterized in that
the nucleic acid molecule is immobilized on the carrier particle via its 5′-terminus by means of bioaffine interactions.

21. Method as claimed in one of the claims 19 to 20,
characterized in that
essentially all nucleotide building blocks of at least two base types carry a fluorescent label.

22. Method as claimed in one of the claims 19 to 21,
characterized in that
the individual nucleotide building blocks are cleaved by an exonuclease.

23. Method as claimed in one of the claims 12 to 17 to synthesize molecules.

24. Method as claimed in claim 23 to synthesize organic molecules or biopolymers.

25. Method as claimed in one of the claims 12 to 24,
characterized in that
the carrier particle is held using a trapping laser.

26. Method as claimed in one of the claims 12 to 25,
characterized in that
the reaction is detected by a confocal fluorescence measurement in a detection volume element.

27. Method as claimed in claim 26,
characterized in that
the detection is carried out by confocal single molecule detection such as fluorescence correlation spectroscopy.

28. Method as claimed in one of the claims 12 to 27,
characterized in that
the detection is by means of a time-resolved decay measurement or time gating in a detection volume element.

29. Method as claimed in one of the claims 12 to 28,
characterized in that
a parallel or/and sequential reaction is carried out on molecules in one or more microchannels.

30. Method as claimed in one of the claims 12 to 29,
characterized in that
the carrier particles are held essentially in the middle of a fluid partial flow and the detection is carried out by conveying reaction products in a laminar flow to a detection volume element which extends over the fluid partial flow in which the carrier particle is held.

31. Method as claimed in claim 30,
characterized in that
the detection volume element is kept as small as possible in order to just be able to detect all reaction products.

32. Method as claimed in one of the claims 12 to 31, comprising transporting the retained carrier particle in the fourth microchannel from a first predetermined position to a second predetermined position which is in the area of the second fluid flow.

33. Method as claimed in one of the claims 12 to 32, comprising changing the flow conditions in the fourth microchannel such that the retained carrier particle comes into the area of the second fluid flow.
